(19) United States
(12) Patent Application Publication
(10) Pub. No.: US 2023/0137614 A1
(43) Pub. Date: May 4, 2023

(54) Cannabinoid Product for Control of Pre-diabetic Markers

(71) Applicant: ECS Brands Ltd., Broomfield, CO (US)

(72) Inventor: Arthur Jaffee, Denver, CO (US)

(73) Assignee: ECS Brands Ltd, Broomfield, CO (US)

(21) Appl. No.: 17/694,910

(22) Filed: Mar. 15, 2022

Related U.S. Application Data
(63) Continuation-in-part of application No. 17/514,387, filed on Oct. 29, 2021.

(51) Int. Cl. A61K 31/352; A61K 31/05; A61K 47/44

(52) U.S. Cl. CPC A61K 31/352; A61K 31/05; A61K 47/44

(57) ABSTRACT

Cannabinoids are increasingly found to have beneficial effects on the human body, even without the intoxicating effects from THC. Herein we describe a cannabinoid which is used to assist with control of pre-diabetic markers in the individual taking the cannabinoid. Tetrahydrocannabivarin, typically but not necessarily combined with assorted cannabinoids, provides a product for control of pre-diabetic markers without the need for diet or exercise.

### CROSS REFERENCE TO RELATED APPLICATION

This application is a continuation-in-part (CIP) of U.S. Ser. No. 17/514,387 filed Oct. 29, 2021 and titled “Cannabinoid Product for Assistance with Weight Loss”.

### BACKGROUND

### Field of the Invention

The present invention relates generally to cannabinoids which assist with control of pre-diabetic markers. More specifically, the present invention relates to varin-rich cannabinoid extracts, such as tetrahydrocannabivarin, and its associated consumption for assist with control of pre-diabetic markers.

### Description of the Related Art

Diabetic mellitus encompasses a group of pancreatic metabolic disorders in which patients present with high blood glucose levels. In recent years, diabetes has become one of the leading causes of death in the United States and worldwide, with incidence increasing each year.

Diabetes is typically categorized in two types, type I and type II. Type I diabetes, previously known as juvenile diabetes, is hereditary and is defined by a lack of sufficient insulin production in the pancreas. Type I diabetics are insulin dependent, which means they require supplemental insulin given by injection multiple times per day. Type I diabetes is not directly associated with obesity or unhealthy lifestyle, though those factors do increase the risk of complications.

Type II diabetes, once called adult-onset diabetes (now known to be untrue), is non-hereditary, and characterized by insufficient insulin production in the pancreas, as well as insulin resistance in the muscle, fat, and liver cells. This results in above average glucose levels in the blood, leading to disorders of the circulatory, nervous, and immune systems. Type II diabetes is exacerbated by numerous health conditions, but the most notable is obesity. Since type II diabetes is not hereditary, it can be reversed by losing weight, maintaining a healthy diet, and regular exercise.

Both types of diabetes are treated similarly, with most patients taking insulin to control their blood glucose levels. During treatment, tests are performed to measure both the point-in-time glucose levels, as well as long-term glucose levels. Instant-read tests report the level of glucose in the blood at a particular instant in time and are used by patients to directly manage their blood glucose levels on a daily basis, particularly after eating or exercising.

Clinicians may require a longer-term viewpoint of blood glucose regulation. One such test for measurement of long-term glucose levels is the glycohemoglobin test, more commonly known as an HbA1c test. When glucose enters the blood stream, it attaches to hemoglobin, a protein found in red blood cells. HbA1c testing works on the principal that glucose will bind to red blood cells at a predictable and well understood rate, and that red blood cells exist in the body for an average of 2-3 months. In combination, this provides clinicians a ‘moving average’ of the blood glucose level for each patient, and typically provides a look-back window between 2-3 months in normal circumstances.

Unlike instant-read tests, HbA1c tests can give insight into long term glucose levels, liver function, and insulin sensitivity, providing a much greater view of the patient's overall glucose metabolism than a simple point-in-time reading. In general, diabetics and pre-diabetics check HbA1c levels 2-4 times a year or more, as directed by their healthcare provider. Keeping HbA1c levels within a target range is critical to lowering chances of serious complications related to diabetes and pre-diabetes. Elevated HbA1c levels, especially those greater than 6.5% (considered diabetic range), are treated as serious conditions that often require intervention to treat. Each patient is different, and complications may present themselves in a variety of ways, most related to the lifestyle, overall health, and type of diabetes.

Since the HbA1c test is ubiquitous in modern medicine, it has become a standard reporting tool for both patients and doctors. In common discussion, it is likely that a diabetic patient may be told to “lower their A1c”; this is due to the nature of the test and what it says about the patient's glucose metabolism. By lowering HbA1c, a patient is effectively reducing their blood glucose level over longer spans of time, indicating that their diabetes is being managed effectively by drugs or lifestyle changes. Thus, a reduction in HbA1c indicates an improvement in glucose metabolism, insulin sensitivity, and/or pancreatic function.

Currently, the most recommended methods for lowering HbA1c are exercise, diet, and maintaining a daily schedule to ensure that diet and exercise are adhered to. Cessation of smoking tobacco products is also frequently recommended to help lower HbA1c.

### SUMMARY

In accordance with the embodiments herein, a cannabinoid product to be consumed to assist with control of pre-diabetic markers is described. The cannabinoid product described herein contains tetrahydrocannabivarin. Other cannabinoids may optionally be included as part of the product. The other cannabinoids are typically cannabidiol, cannabidivarin, cannabigerol, cannabidiolic acid, cannabigerolic acid, or cannabinol.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

In an illustrative embodiment of the invention, the cannabinoid product is either naturally occurring or synthetic tetrahydrocannabivarin (“THCV”) consumed to assist with control of pre-diabetic markers in the person consuming the THCV. In optional embodiments, the THCV may be combined with at least one naturally occurring or synthetic assorted cannabinoids such as cannabidiol (“CBD”), cannabidivarin (“CBDV”), cannabigerol (“CBG”), cannabidiolic acid (“CBDA”), cannabigerolic acid (“CBGA”), and/or cannabinol (“CBN”). Other cannabinoids are contemplated.

In a further embodiment of the invention containing at least one assorted cannabinoid the cannabinoid product is between 1.0 and 95.0 percent by mass of THCV and between 5.0 and 99.0 percent by mass of the at least one assorted cannabinoid. In other embodiments with the assorted cannabinoids, the THCV is typically between 1.0 and 70.0 percent by mass, the CBD is between 25.0 and 50.0 percent by mass, and the remaining assorted cannabinoids are each between 5.0 and 35.0 percent by mass.

Frequently, the invention is within a carrier oil. This carrier oil is typically a hemp oil, such as golden hemp seed oil. Occasionally, the invention would be an emulsion within the carrier oil, however this is not necessary. In an emulsion, water solubility and smaller particle sizes are more ideal for absorption of the invention into the human body. For the invention as an emulsion within the carrier oil, particle sizes between 10.0 nm and 5.0 μm are preferred but not necessary. Larger and smaller particle sizes are contemplated and still allow the invention to function.

Multiple types of delivery systems can be used with the invention. Some embodiments are as an oil, while others are tinctures, gel capsules, liquid emulsions, powders, transdermal preparations, or even as an inhalant. Other types of delivery methods are contemplated.

Most typically, an individual will consume the invention to assist with control of pre-diabetic markers. The use of the invention as a control of pre-diabetic markers supplement has definitive advantages, namely that consumers are not expected to make major changes to their lifestyle or dietary habits when taking the invention. Individuals taking the invention for at least 90 days reported steady control of pre-diabetic markers without dietary restrictions or exercise regimens. By promoting natural, healthy control of pre-diabetic markers, it is expected that participants will be able to integrate themselves into healthier habits without the traditional rebound effects seen in many diet or exercise programs

The clinical trial results related to the invention showed no significant difference in control of pre-diabetic markers response between men and women. It is important to recognize the benefits of control of pre-diabetic markers, regardless of gender, for overall health improvement in overweight individuals. Because the incidence of obesity is nearly equal in men and women, control of pre-diabetic markers should be handled in a way that is equally available and suitable to both men and women. The regulating effects of the invention showed no favoritism toward one gender, and as a result, the invention acts as an effective weight and health management tool for both men and women.

Individuals taking the invention for at least 90 days with no other direct change to lifestyle, including diet and exercise, achieved reduction of pre-diabetic markers by at least 0.2% in HbA1c level compared to the control group. This reduction of pre-diabetic markers lead to a total value of HbA1c of under 5.4% for the individuals taking the invention for at least 90 days.

In another illustrative embodiment, the invention is a cannabinoid product containing hemp seed oil and a mixture of cannabinoids made up of 74.0% CBD, 17.0% CBDV, 5.0% THCV, 2.0% CBG, 1.0% CBDA, 1.0% CBGA, and less than 0.1% CBN, with the percentages of each cannabinoid being percent by mass. This embodiment may be taken to assist with control of pre-diabetic markers. It may be an emulsion; it may be an oil, tincture, gel capsule, liquid emulsion, powder, transdermal preparation, or even as an inhalant. Other forms of manufacture and delivery are contemplated.

What is claimed is:

1. A product, comprising:
tetrahydrocannabivarin; and
wherein the tetrahydrocannabivarin is used to assist with control of at least one pre-diabetic marker in an individual consuming the tetrahydrocannabivarin.

2. The product of claim 1, further comprising:
at least one assorted cannabinoid, wherein the at least one assorted cannabinoid is selected from the group consisting of cannabidivarin, cannabigerol, cannabidiolic acid, cannabigerolic acid, cannabinol, and cannabidiol.

3. The product of claim 2, wherein
the tetrahydrocannabivarin is between 1.0 and 70.0 percent by mass;
the at least one assorted cannabinoid is cannabidiol and at least one additional assorted cannabinoid selected from the same grouping; and
the cannabidiol is between 25.0 and 50.0 percent by mass; and
the at least one additional assorted cannabinoid is between 5.0 and 35.0 percent by mass.

4. The product of claim 2, wherein;
the tetrahydrocannabivarin is between 1.0 and 95.0 percent by mass; and
the at least one assorted cannabinoid is cannabidiol and is between 5.0 and 99.0 percent by mass.

5. The cannabinoid product of claim 1, further comprising a carrier hemp oil.

6. The cannabinoid product of claim 1, wherein the cannabinoid product is prepared in a manner selected from the group consisting of oil, tincture, gel capsule, liquid emulsion, powder, transdermal preparation, and inhalant.

7. The cannabinoid product of claim 1, wherein the cannabinoid product is an emulsion.

8. The cannabinoid product of claim 7, wherein the emulsion has a particle size between 10.0 nm and 5.0 μm.

9. The cannabinoid product of claim 1, wherein the at least one pre-diabetic marker of the individual is HbA1c greater than 5.4%.

10. The cannabinoid product of claim 9, wherein the individual has HbA1c less than 5.4% after taking the cannabinoid product for at least 90 days.

11. A cannabinoid product, comprising:
hemp seed oil; and
a cannabinoid mixture;
wherein the cannabinoid mixture comprises:
cannabidiol at 74.0 percent by mass;
cannabidivarin at 17.0 percent by mass;
tetrahydrocannabivarin at 5.0 percent by mass;
cannabigerol at 2.0 percent by mass;
cannabidiolic acid at 1.0 percent by mass;
cannabigerolic acid at 1.0 percent by mass; and
cannabinol at less than 0.1 percent by mass.

12. The cannabinoid product of claim 11, wherein an individual consumes the cannabinoid product to assist with control of at least one pre-diabetic marker.

13. The cannabinoid product of claim 12, wherein the at least one pre-diabetic marker of the individual is HbA1c greater than 5.4%.

14. The cannabinoid product of claim 12, wherein the individual has HbA1c less than 5.4% after taking the cannabinoid product for at least 90 days.

15. The cannabinoid product of claim 11, wherein the cannabinoid product is prepared in a manner selected from the group consisting of oil, tincture, gel capsule, liquid emulsion, powder, transdermal preparation, and inhalant.

16. The cannabinoid product of claim 11, wherein the cannabinoid product is an emulsion.

17. The cannabinoid product of claim 16, wherein the emulsion has a particle size between 10.0 nm and 5.0 μm.
